(19) United States
(12) Patent Application Publication
(10) Pub. No.: US 2016/0228320 A1
(43) Pub. Date: Aug. 11, 2016

(54) INVERSION THERAPY SPINAL DECOMPRESSION ACCESSORY

(71) Applicant: Robert Mabry, Nashville, TN (US)

(72) Inventor: Robert Mabry, Nashville, TN (US)

(21) Appl. No.: 15/040,859

(22) Filed: Feb. 10, 2016

Related U.S. Application Data
(60) Provisional application No. 62/114,478, filed on Feb. 10, 2015.

(51) Int. Cl. A61H 1/02; A61F 5/042

(52) U.S. Cl. CPC A61H 1/0229; A61F 5/042; A61H 1/0222; A61H 2201/163; A61H 2203/0493

(57) ABSTRACT

An inversion accessory for supporting a user at the waist while using an inversion device. The inversion accessory includes a belt, wherein the belt is configured to be placed around the waist of a user. The inversion accessory also includes one or more rings connected to the belt and a series of bands attached to each of the one or more rings. The inversion accessory further includes a center loop connected to each of the series of bands and a center strap. The center strap is configured to secure to the center loop and attach to the inversion table.

### CROSS-REFERENCE TO RELATED APPLICATIONS

This application claims the benefit of and priority to U.S. Provisional Patent Application Ser. No. 62/114,478 filed on Feb. 10, 2015, which application is incorporated herein by reference in its entirety.

### BACKGROUND OF THE INVENTION

Back pain, especially low-back pain, is a common problem for over 65 million Americans and is the second most reason for doctor's visits. Inversion therapy claims that it is particularly beneficial for the spine in that it relieves pressure on the joints, discs, and nerve roots; this in turn allows discs to return to their original shape, decreasing pressure exerted on the nerve roots. Proponents claim that when the body's weight is upside down, the pull of gravity may decompress the joints of the body below the anchor. Hanging by the feet (inverted) causes each joint in the body to be loaded in an equal and opposite manner to standing in an identical position of joint alignment/load. One such product/example is, Teeter Hang Ups. There are several models to choose from, each offering a conventional method of anchoring the ankles/feet and oscillating the table until the user is inverted. Other examples include: Body Max, Ironman Gravity, and Elite Fitness, all of which are similar in design, differing in cosmetic choices of comfort features.

The problem with this therapy is that by anchoring the ankles/feet, the anchor point is at the most distal aspect of the lower extremity kinetic chain. This only allows for an indirect tractioning of the target area (low-back) and minimal decompression. Other contraindications are lower extremity joint dysfunction i.e. arthritis, prosthetics, post-surgery syndromes, high blood pressure, diabetic retinopathy etc. would prohibit inversion.

### BRIEF SUMMARY OF SOME EXAMPLE EMBODIMENTS

This Summary is provided to introduce a selection of concepts in a simplified form that are further described below in the Detailed Description. This Summary is not intended to identify key features or essential characteristics of the claimed subject matter, nor is it intended to be used as an aid in determining the scope of the claimed subject matter.

One example embodiment includes an inversion accessory for supporting a user at the waist while using an inversion device. The inversion accessory includes a belt, wherein the belt is configured to be placed around the waist of a user. The inversion accessory also includes one or more rings connected to the belt and a series of bands attached to each of the one or more rings. The inversion accessory further includes a center loop connected to each of the series of bands and a center strap. The center strap is configured to secure to the center loop and attach to the inversion table.

Another example embodiment includes system for decompressing a user's spine. The system includes an inversion device, the inversion device configured to hold the user in a head down position. The system also includes an inversion accessory. The inversion accessory includes a belt, wherein the belt is configured to be placed around the waist of a user. The inversion accessory also includes one or more rings connected to the belt and a series of bands attached to each of the one or more rings. The inversion accessory further includes a center loop connected to each of the series of bands and a center strap. The center strap is configured to secure to the center loop and attach to the inversion table.

Another example embodiment includes system for decompressing a user's spine. The system includes an inversion table, the inversion table configured to hold the user in a head down position. The system also includes an inversion accessory. The inversion accessory includes a belt, wherein the belt is configured to be placed around the waist of a user. The inversion accessory also includes a set of four rings connected to the belt and a center loop. The inversion accessory further includes a first band. The first band attaches to the first ring in the set of four rings, passes through the center loop and attaches to the second ring in the set of four rings. The inversion accessory moreover includes a second band, wherein the second band attaches to the third ring in the set of four rings, passes through the center loop and attaches to the fourth ring in the set of four rings. The inversion accessory additionally includes a center strap. The center strap is secured to the center loop and attached to the inversion table.

These and other objects and features of the present invention will become more fully apparent from the following description and appended claims, or may be learned by the practice of the invention as set forth hereinafter.

### BRIEF DESCRIPTION OF THE DRAWINGS

To further clarify various aspects of some example embodiments of the present invention, a more particular description of the invention will be rendered by reference to specific embodiments thereof which are illustrated in the appended drawings. It is appreciated that these drawings depict only illustrated embodiments of the invention and are therefore not to be considered limiting of its scope. The invention will be described and explained with additional specificity and detail through the use of the accompanying drawings in which:

FIG. 1 illustrates a system for performing inversion therapy;

FIG. 2A illustrates a perspective view of an example of an inversion accessory; and

FIG. 2B illustrates a close up view of an example of an inversion accessory.

### DETAILED DESCRIPTION OF SOME EXAMPLE EMBODIMENTS

Reference will now be made to the figures wherein like structures will be provided with like reference designations. It is understood that the figures are diagrammatic and schematic representations of some embodiments of the invention, and are not limiting of the present invention, nor are they necessarily drawn to scale.

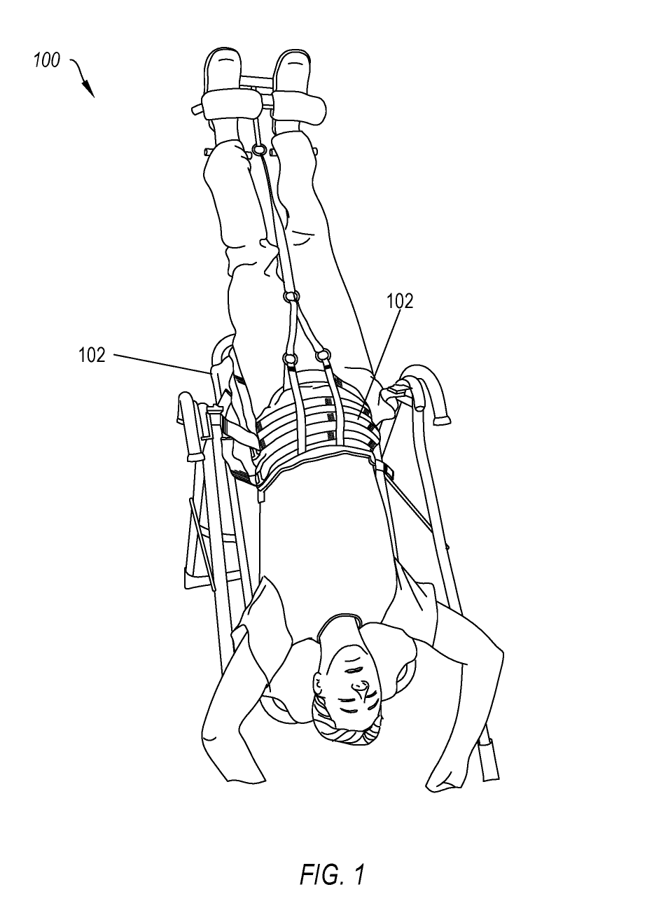

FIG. 1 illustrates a system 100 for performing inversion therapy. Inversion therapy involves hanging upside down or at an inverted angle with the intention of therapeutic benefits. The process of doing so is called inversion. It is a form of spinal decompression and is a form of spinal traction. When the body's weight is suspended from the lower body the pull of gravity may decompress the joints of the body below the anchor. Hanging by the feet, as with gravity boots or inversion tables or inversion chairs, causes joints of the back to be stretched and expanded. Inversion therapy of this sort is often a relief for back pain.

When the body's weight is suspended from the lower body—rather than borne on the hands as in handstands or headstands or hanging from a bar with arms at sides, which are also forms of inversion—the pull of gravity may decompress the joints of the body below the anchor. Hanging by the feet, as with gravity boots or inversion tables, causes each joint in the body to be loaded in an equal and opposite manner to standing in an identical position of joint alignment. Inversion therapy may be particularly beneficial for the spine in that it relieves pressure on the discs and nerve roots which in turn allows discs to recover lost moisture and to return to their original shape, decreasing the pressure they can exert on nerves.

In addition, inversion may be a tool to restore proper alignment to the spine, which may assist in maintaining proper posture when later righted. As misalignment of the hips and spine can itself be a source of inflammation and pain, this is another benefit of inversion as a back therapy.

Further, inversion may be effective in gaining flexibility. Static-active stretching methods impossible to perform while standing can be performed upside down for the spinal flexors, side flexors and extensors, and sit-ups are a closed-chain exercise for the hip flexors (a static-active stretch for the hip extensors) compared to the upright exercise, leg raises, which are open-chain movements. Due to increased spacing in the joint which can occur in response to traction, the muscles crossing that joint are pre-stretched, and as such, experience a greater lengthening compared to the equivalent joint angle while under no traction (lying down) or while being compressed (standing).

Moreover, inversion may stimulate circulation differences due to gravity acting on the circulatory system in an opposite manner. I.e., while a user is inverted gravity opposes what it would normally assist, and assists what it would normally oppose compared to standing upright. This pooling of blood and greater circulation is thought to increase oxygen flow to those tissues, primarily in the brain or roots of the hair.

FIG. 1 shows that the system 100 can include an inversion device 102. The inversion device 102 is any mechanism that allows the user to invert. For example, the inversion device 102 can include gravity boots, a gravity chair, an inversion table or other means for providing inversion therapy. Gravity boots are the most aggressive form of the therapy and put the body completely upside down. Inversion tables are typically flat in shape and will allow the user to adjust from being upright to being horizontal and completely upside down. Tables allow for more control than boots because the user can choose a smaller angle, such as 15 degrees from horizontal, to stretch the user's back, which puts less strain on the user's feet, ankles, knees, and other joints. Inversion chairs are like tables, but they take most of the pressure off the ankles and feet. The user straps into a chair and rotates backwards to a comfortable position.

FIG. 1 also shows that the system 100 can include an inversion accessory 104. The inversion accessory 104 can be used in conjunction with the inversion device 102. The inversion accessory 104 supports a user at the waist during inversion therapy, rather than another location, such as the user's feet or legs. That is, the inversion accessory 104 converts the anchor point form the user's legs or feet to the user's waist. This allows a user to receive the benefits of spinal decompression without causing damage to the user's legs. In particular, since the user's legs are no longer supporting the user's weight no additional pressure is placed on the user's legs or feet.

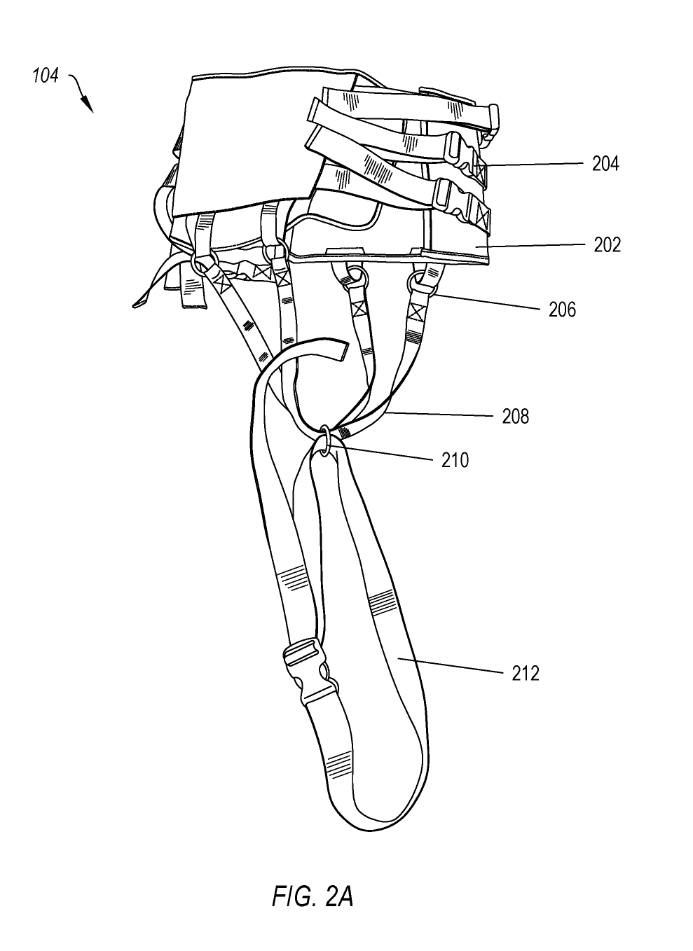

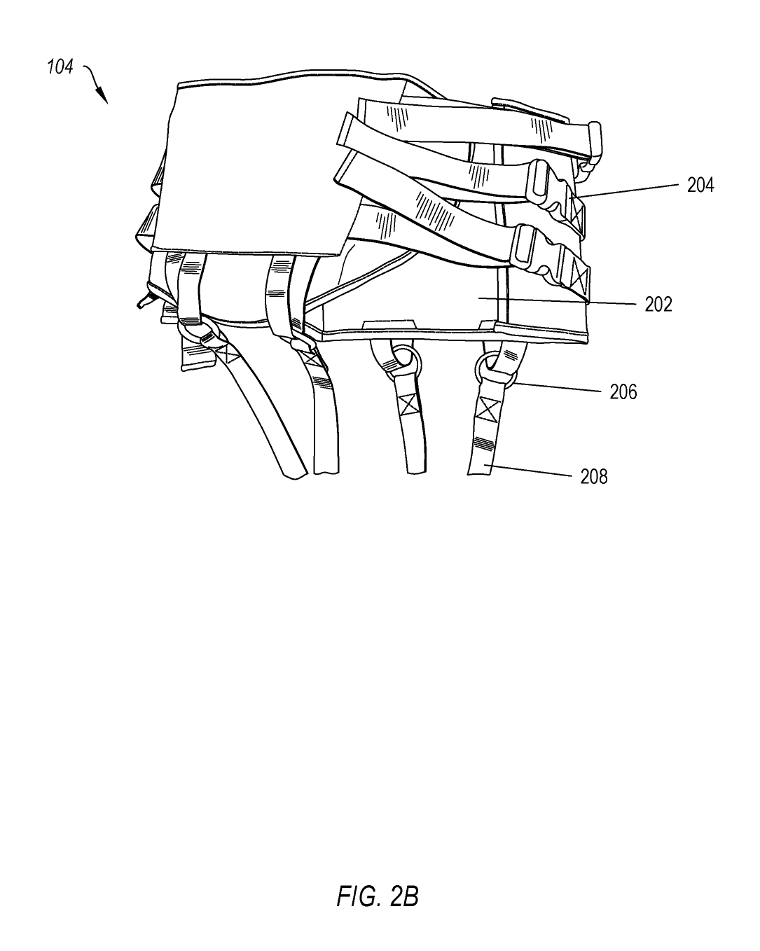

FIGS. 2A and 2B (collectively “FIG. 2”) illustrate an example of an inversion accessory 104. FIG. 2A illustrates a perspective view of an example of an inversion accessory 104; and FIG. 2B illustrates a close up view of an example of an inversion accessory 104. The inversion accessory 104 supports a user's waist during inversion therapy. Gravity assisted traction is quantifiable in that the amount of traction applied is determined by two factors. The user's body mass, a relative constant and gravity, a constant. The inversion table, independent of the manufacturer, relies on these factors. As stated above the inherent flaw with standard inversion is that the contact point (ankles) is at the most distal point in relation to the target (pelvis). This is very inefficient. The inversion accessory 104 puts the contact point directly on the target. Thus allowing for closer to 100% of the traction to be focused on its intention, decompressing the spine. There are contraindications to inverting, mentioned above, such as orthopedic prosthetics like knee and hip replacement. The inversion accessory 104, by making the contact point directly on the target helps to eliminate this by dramatically limiting the amount of tension being placed on these joints. The inversion accessory 104 also makes the overall experience of inverting more comfortable by reducing the tension on the joints of the lower extremity

FIG. 2 shows that the inversion accessory 104 can include a belt 202 or harness. The belt 202 can be placed around the user and attached to the inversion table. The tension supplied by gravity can then be applied to the user at the location of the belt 202. In particular, the belt 202 is secured around the waist of the user and the weight of the user is transferred to an inversion device by the belt 202 through the user's weight. The belt 202 can be made of any desired material, such as nylon or polyester and can include padding or other materials to increase the comfort of the user. For example, the belt 202 can include two panels constructed of nylon canvas backing with a neoprene inner lining. The neoprene lining functions to resist slipping when the inversion accessory 104 is in use. This allows the belt to stay in place on the target while in use and maximizing the desired benefit.

FIG. 2 also shows that the inversion accessory 104 can include a securing mechanism 204. The securing mechanism 204 is configured to secure the belt 202 about the waist of the user. I.e., the securing mechanism 204 ensures that the inversion accessory 104 does not move relative to the user. If the securing mechanism 204 does permit motion of the inversion accessory 104 relative to the user then the user can slip during inversion. Therefore, the securing mechanism must be adjustable to the user. For example, the inversion accessory 104 can include straps and buckles or clips which can be releasably attached to rings on the belt 202. That is, the belt 202 can be placed around the waist of the user and secured using the straps and buckles or clips.

As used in the specification and the claims, the phrase “configured to” denotes an actual state of configuration that fundamentally ties recited elements to the physical characteristics of the recited structure. That is, the phrase “configured to” denotes that the element is structurally capable of performing the cited element but need not necessarily be doing so at any given time. As a result, the phrase “configured to” reaches well beyond merely describing functional language or intended use since the phrase actively recites an actual state of configuration.

Multiple straps can allow for better customization and/or multiple pressure points. I.e., multiple straps allow the belt 202 to make contact at several points with the waist of the user. For example, the securing mechanism 204 can include two or three straps. This not only enhances the effect of the traction applied (making it more specific to the target) but also makes the inversion accessory 104 virtually a one size fits all. Likewise, the buckles can be releasable to allow a user to more easily secure the straps. The buckle or clasp is a device used for fastening two loose ends, with one end attached to it and the other held by a catch in a secure but adjustable manner. For example, the buckles can include side release buckles or any other fastening mechanism.

FIG. 2 further shows that the inversion accessory 104 can include a series of rings 206 attached to the belt 202. The series of rings 206 allow a connection between the belt 202 and an inversion device. That is, the weight of the user is transferred from the belt 202 to the inversion device through the series of rings 206. There may be enough rings to avoid causing any pressure points on the user. For example, the series of rings 206 can include two rings on the front of the belt 202 and two rings on the back of the belt 202. The series of rings 206 can include any material of sufficient strength, such as metal, to support the weight of a user. The series of rings 206 can be directly attached to the belt 202 or can be attached to fabric loops on the belt 202.

FIG. 2 additionally shows that the inversion accessory 104 can include bands 208 attached to the series of rings 206. The bands 208 support the user's weight via the belt 202 and the series of rings 206. The bands 208 can include any material that is of sufficient strength to support the weight of the user. For example, the bands 208 can be made of nylon or any other desired material.

FIG. 2 moreover shows that the inversion accessory 104 can include a center loop 210. The bands 208 attach to the center loop 210. For example, the bands 208 can attach directly to the center loop 210 via buckles, clips or any other mechanism. Additionally or alternatively, the bands 208 can pass through the center loop 210. For example, a band 208 can attach to a front ring in the series of rings 206 pass through the center loop 210 then attach to a rear ring in the series of rings 206. Thus, when the user is inverted and tension is placed on the center loop 210 the tension is transferred to the belt 202 via the bands 208.

FIG. 2 also shows that the inversion accessory 104 can include a center strap 212. The center strap 212 connects the center loop 210 to the inversion device. The center strap 212 can adjust to the height of the user. For example, the center strap 212 can be looped around the ankle bracket post of an inversion table. The user bends at the knees and adjusts the center strap 212 via the buckle thereby taking out the slack from the center strap 212. The user inverts as they would normally, but instead of the tension from the gravity assisted traction coming from the ankles, closer to 100% of this force is now coming from the inversion accessory 104.

Once on the inversion table the table rotates, inverting the user, so that gravity pulls the user down while the inversion accessory 104 is secured to the table. This causes direct decompression of the user's lumbar spine (low-back). This allows users who were unable to utilize inversion therapy because of contraindications previously listed to benefit from this decompression inversion accessory 104 without having to be in full inversion. Greater, more direct decompression occurs by utilizing the inversion accessory 104 around the waist focusing the decompression on the back. The user may also benefit from inverting as normal with the added benefit of direct low-back decompression.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. An inversion accessory for supporting a user at the waist while using an inversion device, the inversion accessory comprising:
a belt, wherein the belt is configured to be placed around the waist of a user;
one or more rings connected to the belt;
a series of bands attached to each of the one or more rings;
a center loop connected to each of the series of bands;
a center strap, the center strap configured to:
secure to the center loop; and
attach to the inversion table.

2. The inversion accessory of claim 1, further comprising:
a securing mechanism, the securing mechanism configured to secure the belt around the waist of the user.

3. The inversion accessory of claim 2, wherein the securing mechanism includes:
one or more straps.

4. The inversion accessory of claim 3, wherein the securing mechanism includes:
two straps.

5. The inversion accessory of claim 1, wherein the securing mechanism includes:
a buckle on each of the one or more straps.

6. The inversion accessory of claim 5, wherein the buckles include:
side release buckles.

7. The inversion accessory of claim 1, wherein at least one of the series of bands:
attaches to a first ring of the one or more rings attached to the belt;
passes through the center loop; and
attaches to a second ring of the one or more rings attached to the belt.

8. The inversion accessory of claim 1, wherein the belt includes a non-slip inner surface.

9. The inversion accessory of claim 1, wherein the belt includes one or more panels.

10. The inversion accessory of claim 1, wherein the one or more panels include a nylon covering.

11. The inversion accessory of claim 1, wherein the one or more panels includes a neoprene inner lining.

12. The inversion accessory of claim 1, wherein the band in the one or more bands include:
nylon.

13. A system for decompressing a user's spine, the system comprising:
an inversion device, the inversion device configured to hold the user in a head down position; and
an inversion accessory, the inversion accessory including:
a belt, wherein the belt is configured to be placed around the waist of the user;
one or more rings connected to the belt;
a series of bands attached to each of the one or more rings;
a center loop connected to each of the series of bands;
a center strap, the center strap configured to:
secure to the center loop; and
attach to the inversion table.

14. The system of claim 13, wherein the inversion device includes:
gravity boots.

15. The system of claim 13, wherein the inversion device includes:
an inversion table.

16. The system of claim 13, wherein the inversion device includes:
an inversion chair.

17. A system for decompressing a user's spine, the system comprising:
an inversion table, the inversion table configured to hold the user in a head down position; and
an inversion accessory, the inversion accessory including:
a belt, wherein the belt is secured around the waist of the user;
a set of four rings connected to the belt;
a center loop;
a first band, wherein the first band:
attaches to the first ring in the set of four rings;
passes through the center loop; and
attaches to the second ring in the set of four rings;
a second band, wherein the second band:
attaches to the third ring in the set of four rings;
passes through the center loop; and
attaches to the fourth ring in the set of four rings;
a center strap, the center strap:
secured to the center loop; and
attached to the inversion table.

18. The system of claim 17, wherein the length of the center strap is adjustable.

19. The system of claim 18, wherein the center strap includes a side release buckle.

20. The system of claim 17, wherein the center strap passes through the center loop.
